(19) United States
(12) Patent Application Publication
(10) Pub. No.: US 2019/0021899 A1
(43) Pub. Date: Jan. 24, 2019

(54) URINARY INCONTINENCE DEVICE

(71) Applicant: Torfrieda Alma Vlet, Union, NJ (US)

(72) Inventor: Torfrieda Alma Vlet, Union, NJ (US)

(21) Appl. No.: 15/652,480

(22) Filed: Jul. 18, 2017

(51) Int. Cl. A61F 5/453; A61F 2/00

(52) U.S. Cl. CPC A61F 5/453; A61F 2/0009

(57) ABSTRACT

A urinary incontinence device is provided. The incontinence device may generally be comprised of a pouch having an absorbent material therein. An outer material, that is impervious to liquid, retains the absorbent material as well as any liquid excreted by the patient. The device may be further used in conjunction with a waist strap or harness to further support the device.

### CLAIM OF PRIORITY

This application claims priority to U.S. Application 62/363,628 filed on Jul. 18, 2016, the contents of which is herein fully incorporated by reference in its entirety.

### FIELD OF THE EMBODIMENTS

The field of the embodiments of the present invention is related to devices for urinary assistance and/or incontinence. In particular, the embodiments provide for a superior to traditional catheters.

### BACKGROUND OF THE EMBODIMENTS

A wide variety of incontinence products have been developed for the incontinent adult. Incontinence pads, diapers, and other garments have been used for many years to alleviate the practical and hygienic problems encountered by individuals suffering from incontinence issues. Normally, these diapers have one or more inner layers of absorbent material contained in an outer layer of waterproof fabric, so that any liquid (urine) excreted by the patient is retained by the diaper and prevented from leaking outside the areas of contact with the patient's body. However, as many of such devices allow for contact between a patient's skin and wet material, skin irritation, ashes, and the like are a common afflictions of the wearer.

Further, the absorbent material within the diaper is typically incapable of adequately rapidly absorbing a high flow rate of voided urine. Consequently, the urine will often overflow the diaper, resulting in the wetting of the wearer's clothes. Additionally, these diapers are best suited for women where the diaper bag opening can be located proximate the urethra to properly receive urine. These diapers are a problem for males wherein the position of the urethra outlet may not directed into the ideal position at the time of voiding.

In other instances, catheters may be employed to drain urine from the bladder from patients. However catheters can be extremely uncomfortable and cause numerous painful side effects. For example, men sometimes have bladder spasms while the catheter is in their penis. These spasms may be caused by the catheter bothering the bladder. In other instances, there may be blood in the urine. It is normal for the urine draining from your catheter to have some small blood clots. In addition, infections of the urinary tract are very common. These are only side effects caused by the catheter, whereas one must also keep collection bag clean and prevent tugging on the catheter save for causing potentially much more serious issues.

Thus, there is a need for a comfortable and effective urine retainment device. Such a device should rapidly absorb urine and minimize contact of the skin with the urine. The present invention and its embodiments meets and exceeds these objectives.

### Review of Related Technology:

U.S. Pat. No. 6,569,135 pertains to a urine absorbent pouch for male incontinence which completely surrounds the patient's penis, thereby minimizing the patient's exposure to wetness. The pouch is constructed from a piece of material which has a hole portion and a continuous portion, the hole portion containing a hole which is adapted to receive a patient's penis. A plurality of sealing means are located along the edges of the hole portion so when the sealing means are sealed onto the continuous portion, there is minimal contact between the sealing means and the patient. Additionally, a urine absorbent pouch for male incontinence can be created from a commercially available baby diaper by cutting a hole in the diaper and attaching additional sealing means to the portion of the diaper with the hole.

U.S. Pat. No. 5,094,853 pertains to a novel arthropodicidally-active composition-of-matter. Such comprises an aqueous lower alkanol solvent, a toxicant contained within the solvent, and an effective amount of an emulsifier, also contained within the solvent, for forming a foam matrix of predetermined stability. Also disclosed are methods for producing such a composition-of-matter.

U.S. Pat. No. 4,559,051 pertains to a disposable diaper having improved liquid receiving and retaining capabilities. The diaper includes a waterproof barrier formed as a flattened bag and having a single opening located adjacent the perineal area of the body of the wearer. Two layers of filler material are positioned within the barrier bag. The upper layer adjacent the opening is a wicking material, while the lower layer is a superabsorbent material. In a male version of the diaper, a pouch is formed over the opening into which the wearer's penis is inserted to direct voided urine through the bag opening.

Various devices are known in the art. However, their structure and usage are substantially different from the present disclosure. The other inventions also fail to solve all the problems taught by the present disclosure. At least one embodiment of this invention is presented in the drawings below and will be described in more detail herein.

### SUMMARY OF THE EMBODIMENTS

Generally the embodiments of the present invention provide for a male incontinence device that may be worn by a user as needed, such as, before, during, or after surgery or by those generally having incontinence issues. The device generally comprises a pouch lined or filled with an absorbent material and lined (on the outside) with a liquid impermeable material. The pouch has an opening for inserting of a male penis. The opening maybe secured with flaps to prevent the pouch from falling, slipping, or otherwise becoming separated from the user.

In some instances, a support belt or harness is used to provide additional support to the pouch. Such a belt may wrap around a waist of a user and be connected strategically to the pouch to alleviate any stresses placed on the penis by the pouch. In yet other instances, the pouch has an adhesive patch thereon such that the pouch can be adhered to a skin or clothing surface to achieve a similar result to the belt. The adhesive may be used singularly or in conjunction with the belt.

In one embodiment of the present invention there is a urinary incontinence device having a pouch with an inner surface and an outer surface defining an opening, wherein the inner surface is comprised of an absorbent material, and wherein the outer surface is made of a liquid impervious material; at least one securement mechanism coupled to the pouch; and at least one retainment mechanism configured to be coupled to the pouch and a user of the pouch.

In another embodiment of the present invention there is a male urinary incontinence device having a pouch with an inner surface and an outer surface defining an opening with the opening being configured to receive a human penis, wherein the inner surface is comprised of an absorbent material, and wherein the outer surface is made of a liquid impervious material; two securement mechanisms coupled to the pouch, wherein each of the two securement mechanisms comprises, a first end and a second end with each of the first end and the second end coupled to the pouch thereby forming a loop; and a length of material having a first end and a second end with the first end being configured to be coupled to one of the two securement mechanisms and the second end being configured to be coupled to another of the two securement mechanisms.

In yet another embodiment of the present invention there is a male urinary incontinence device having a pouch with an inner layer and an outer layer defining an opening with the opening being configured to receive a human penis, wherein the inner layer is comprised of sodium polyacrylate, and wherein the outer layer is made of a petroleum based material; at least one flap disposed in proximity to the opening and being configured to adjust the size of the opening, wherein a securement mechanism is disposed on one end of the at least one flap to secure a position of the at least one flap; two securement mechanisms coupled to the pouch, wherein each of the two securement mechanisms comprises, a first end and a second end with each of the first end and the second end coupled to the pouch thereby forming a loop; an adhesive material being disposed on an outer layer of the pouch; and a length of material having a first end and a second end with the first end being configured to be coupled to one of the two securement mechanisms and the second end being configured to be coupled to another of the two securement mechanisms.

In general, the present invention succeeds in conferring the following, and others not mentioned, benefits and objectives.

It is an object of the present invention to provide a device that removes the need for a traditional catheter.

It is an object of the present invention to provide a device that prevents spillage or urine and/or other bodily fluids.

It is an object of the present invention to provide a device that is easy to put on and take off.

It is an object of the present invention to provide a device that is disposable.

It is an object of the present invention to provide a device that is a one size fits all device.

It is an object of the present invention to provide a device that is lightweight and comfortable.

It is an object of the present invention to provide a device that allows a user to be mobile while using the device.

It is an object of the present invention to provide a device that can be secured or otherwise supported to provide additional comfort to the user.

### BRIEF DESCRIPTION OF THE DRAWINGS

FIG. 1 is a front perspective view of a pouch of an embodiment of the present invention.

FIG. 2 is a front perspective view of a pouch with an attached support of the present invention.

FIG. 3 is a cutaway side view of a pouch of an embodiment of the present invention.

FIG. 4 is a front perspective view of an embodiment of the present invention in use.

### DETAILED DESCRIPTION OF THE EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification that various modifications and variations can be made thereto.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

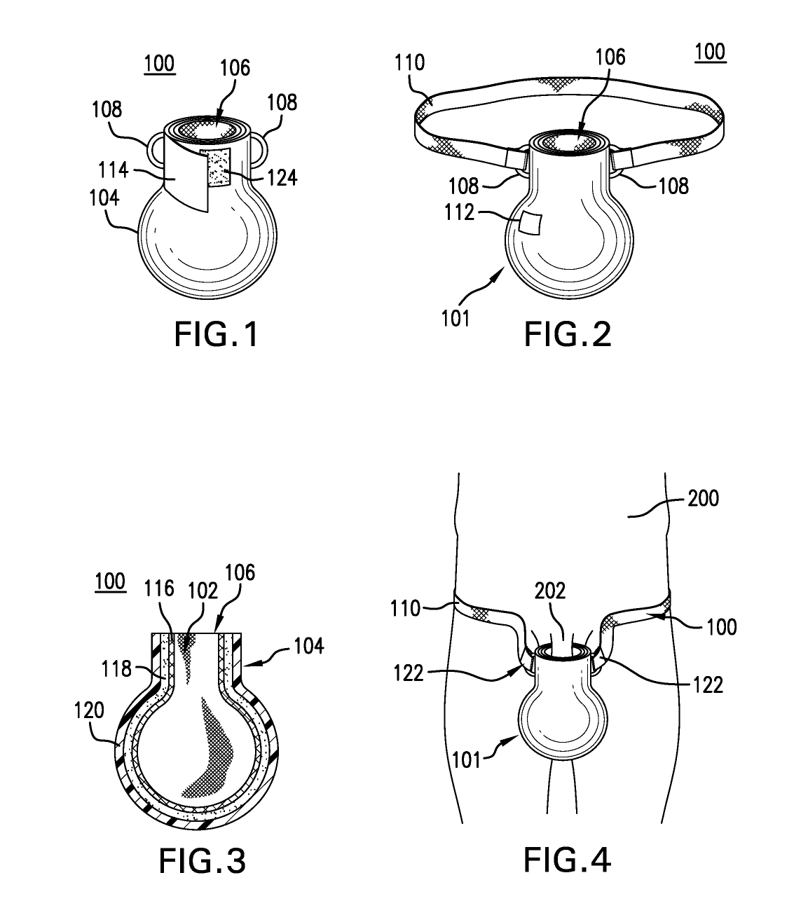

Referring now to FIGS. 1-2, there is a first view of the device 100. Generally, the device 100 has an opening 106, securement mechanisms 108, attachment mechanism 124, flap 114, outer surface 104, retainment mechanism 110, and adhesive area 112.

The device 100 is shaped to form a pouch 107 having an inner surface 102 (see FIG. 3) and an outer surface 104. Between the inner surface 102 and the outer surface 104 are a number of layers of various materials. The number of layers may vary and may have about one layer to about ten layers, and more preferably may have about two layers to about four layers. Generally, the inner layer(s) comprise an absorbent material and the outer layer comprises a liquid impervious material. The pouch 107 may have an overall length of about 3 inches to about 12 inches. The pouch 107 has a circumference of about 8 inches.

The pouch 107 has an opening 106 which is sized to be configured to receive a human penis. The opening 106 may be adjustable by way of at least one, and in some embodiments, two flaps 114. In other embodiments, there is an elastic material that surrounds the opening 106 that is configured to accommodate up to 10 inches in diameter allowing for easy application and removal of the device 100.

The flap(s) 114 have a positioning material 124 on a surface thereof and there is, in some embodiments, a complementary positioning material 127 disposed on the outer surface 104 of the device 100. By tightening or pulling on the flaps 114 and coupling the flaps 114 to the pouch 107 the size of the opening can effectively be changed. This allows the opening to accommodate a penis of varying sizes while remaining securely attached to the user. A base or lower area of the pouch 107 is wider than a neck area of the pouch 107 with the neck area being in proximity to the opening 106. The neck area of the pouch 107 may be comprised of differing or additional materials configured to be comforting to a user of the device 100. It is intended that the neck area will have a length configured to accommodate a shaft of the penis inserted therein. This allows for the user to minimize any contact with the “collection” area of the pouch 107 as it begins to accumulate liquid waste therein.

Securement mechanisms 108 are coupled to an outer surface 104 of the device 100. Preferably these securement mechanisms 108 are coupled to the neck area of the pouch 107, however, the securement mechanisms 108 may be coupled anywhere on the outer surface 104 of the device 100. In at least one embodiment, the securement mechanisms 108 comprise a first end and a second end with each of the first end and the second end being coupled to the device 100 thereby forming a loop. In yet another embodiment, the securement mechanisms 108 are elastic in nature. In a preferred embodiment, there are two securement mechanisms 108 with one securement mechanism 108 being coupled to the device 100 on an opposing side of the device 100. In another embodiment there is only one securement mechanism 108 disposed on the device 100.

A retainment mechanism 110 is utilized to further secure the pouch 107 to a user. Here, the retainment mechanism 110 takes the form of a belt. The retainment mechanism 110 is further size adjustable to accommodate a waist area of a user. As various users can fluctuate from one to another in size, a fastening mechanism, such as a hook and loop fastener, may be utilized to provide differing sizes of the retainment mechanism 110. Further, each of a first end and a second end of the retainment mechanism 110 is coupled to one of the two securement mechanisms 108. This forms a complete device 100 consisting primarily of the pouch 107 and the retainment mechanism 110.

An adhesive area or patch 112 may further be present on an outer surface 104 of the device. In some embodiments, a non-stick covering may be present on the adhesive area 112 until it is desired to utilize the adhesive area 112. In some embodiments, the adhesive area 112 is on a front of the device, whereas in other embodiments it is on the side or rear of the device. In other embodiments, the adhesive area 112 is present at more than one location on the device 100. Generally, when desired, the adhesive area 112 can be exposed allowing the adhesive to be affixed to an article of clothing of the user. In other embodiments the adhesive area 112 may be coupled directly to a skin surface of the user. This provides an additional point of contact to alleviate any strain or stress placed on the device 100 or the user as the device 100 becomes saturated. The adhesive area 112 may be utilized in conjunction with the retainment mechanism 110 or may be utilized solely on its own.

Referring now to FIG. 3, a sectional side view of the device 100, namely the pouch 107, is shown. Here, the layers of the pouch 107 are visible, as are the interrelationships between those layers. The pouch 107 generally has an inner surface 102, an absorbent layer 118, a cloth layer 116, an outer surface 104, and a petroleum based (plastic) layer 120.

In use, the outer surface 104 comprises a liquid impervious material. The outer surface 104 abuts the plastic layer 120 and may be integrated with the plastic layer 120. In some embodiments, the outer surface 104 is simply an outer surface of the plastic layer 120. The plastic layer 120 is preferably about 0.5 inches to about 3 inches in thickness. In yet other embodiments, this outer surface 104 is a distinct and separate layer.

The inner surface 102 comprises a liquid absorbent material and abuts a cloth layer 116. The inner surface 102 may be integrate with the cloth layer 116. In some embodiments, the inner surface 102 is simply an inner surface of the cloth layer 116. In yet other embodiments, this is a distinct and separate layer. Between the plastic layer 120 and the cloth layer 116 is an absorbent layer 118. The absorbent layer 118 is preferably comprised of sodium polyacrylate, but may be comprised of a blend of materials configured to readily absorb moisture. In at least one embodiment, it is desired that the absorbent layer 118 is capable of absorbing up to 2 L of fluid and more preferably up to 1 L of fluid.

In other embodiments, the absorbent layer 118 and the cloth layer 116 are separable from the plastic layer 120. In some instances, an additional layer, between the absorbent layer 118 and the plastic layer 120 exists such that this removal is facilitated without losing the liquid collected therein. In yet other embodiments, additional absorbent material may be disposed on the cavity formed in the pouch 107. However, such material may be provided for comfort purposes as generally it is desired to pull moisture away from the skin surface.

FIG. 4 illustrates a preferred implementation of an embodiment of the present invention. Here, the device 100 is shown coupled to a user 200. In practice, the penis 202 of the user 200 is inserted in the opening 106 (see FIG. 1). The retainment mechanism 110 is disposed around a waist of the user 200 and the fastening mechanism(s) 122 is manipulated such that the device 100 is held in place. The device 100 can then be held in place until it has become saturated and needs to be changed or until it is otherwise desirable to remove the device 100.

The embodiments of the present invention described herein provide for a male incontinence device that may be worn by a user as needed, such as, before, during, or after surgery or by those generally having incontinence issues. Such a device is intended to replace traditional catheters which can be painful to use and can cause serious infections amongst other problems.

What is claimed is:

1. A urinary incontinence device comprising:
a pouch having at least one inner layer and an outer layer defining an opening,
wherein one of the at least one inner layer is comprised of an absorbent material, and
wherein the outer layer is made of a liquid impervious material;
at least one securement mechanism coupled to the pouch; and
at least one retainment mechanism configured to be coupled to the pouch.

2. The device of claim 1 wherein a size of the opening is adjustable.

3. The device of claim 1 wherein the securement mechanism comprises a first end and a second end with each of the first end and the second end coupled to the pouch thereby forming a loop.

4. The device of claim 1 further comprising an adhesive disposed on an outer surface of the pouch.

5. The device of claim 1 wherein the absorbent material contains sodium polyacrylate.

6. The device of claim 1 wherein there are two securement mechanisms.

7. The device of claim 6 wherein the at least one retainment mechanism is coupled to each of the two securement mechanisms.

8. The device of claim 2 further comprising flaps disposed in proximity to the opening and being configured to adjust the size of the opening.

9. The device of claim 1 wherein the opening is configured to receive a human penis.

10. A male urinary incontinence device comprising:
a pouch having an inner surface and an outer surface defining an opening with the opening being configured to receive a human penis,
wherein an absorbent material is disposed between the inner surface and the outer surface, and
wherein the outer surface is made of a liquid impervious material;
two securement mechanisms coupled to the pouch,
wherein each of the two securement mechanisms comprises,
a first end and a second end with each of the first end and the second end coupled to the pouch thereby forming a loop; and
a length of material having a first end and a second end with the first end being configured to be coupled to one of the two securement mechanisms and the second end being configured to be coupled to another of the two securement mechanisms.

11. The device of claim 10 wherein the length of material has a fastening mechanism disposed on each of the first end and the second end of the length of material.

12. The device of claim 10 wherein the outer surface comprises an outer layer and an inner layer with the outer layer comprised of the liquid impervious material and the inner layer being a cloth layer.

13. The device of claim 10 wherein the device is configured to hold up to 1 L of fluid.

14. A male urinary incontinence device comprising:
a pouch having at least one inner layer and an outer layer defining an opening with the opening being configured to receive a human penis,
wherein the at least one inner layer is comprised of sodium polyacrylate, and
wherein the outer layer is made of a petroleum based material;
at least one flap disposed in proximity to the opening and being configured to adjust the size of the opening,
wherein a positioning mechanism is disposed on one end of the at least one flap to secure a position of the at least one flap;
two securement mechanisms coupled to the pouch,
wherein each of the two securement mechanisms comprises,
a first end and a second end with each of the first end and the second end coupled to the pouch thereby forming a loop;
an adhesive material being disposed on an outer layer of the pouch; and
a length of material having a first end and a second end with the first end being configured to be coupled to one of the two securement mechanisms and the second end being configured to be coupled to another of the two securement mechanisms,
wherein the length of material has a fastening mechanism disposed on each of the first end and the second end of the length of material.

15. The device of claim 15 wherein the adhesive material is disposed on a back surface of the pouch.

16. The device of claim 15 wherein the inner layer is removable.

17. The device of claim 15 wherein the outer layer of the pouch is about 3 inches thick.

18. The device of claim 15 wherein the fastening mechanism is a hook and loop fastener.

19. The device of claim 15 wherein a base of the pouch is wider than the opening of the pouch.

20. The device of claim 15 wherein the two securement mechanisms are elastic.
